(19) United States
(12) Patent Application Publication
(10) Pub. No.: US 2006/0116330 A1
(43) Pub. Date: Jun. 1, 2006

(54) Methods of mimicking the metabolic effects of caloric restriction by administration of mannoheptulose

(75) Inventors: Josef Pitha, Rockville, MD (US); George Roth, Pylesville, MD (US); Michael Griffin Hayek, Dayton, OH (US); Stefan Patrick Massimino, Kettering, OH (US); Michael Anthony Ceddia, Newburgh, IN (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignees: The Iams Company, Cincinnati, OH (US); GeroTech, Inc., Pylesvillle, MD (US)

(21) Appl. No.: 11/313,199

(22) Filed: Dec. 20, 2005

Related U.S. Application Data
(63) Continuation-in-part of application No. 09/950,052, filed on Sep. 12, 2001.
Application No. 09/950,052 is a continuation-in-part of application No. 08/889,877, filed on Jul. 8, 1997, now abandoned.

(51) Int. Cl. A61K 31/70; A61K 31/045

(52) U.S. Cl. 514/23; 514/738

(57) ABSTRACT

Disclosed herein are methods of using glucose anti-metabolites to alter utilization of glucose or other energy sources and to mimic metabolic effects of caloric restriction. In particular, in one embodiment herein, methods of enhancing longevity in an animal are described, the methods comprising administration of a composition comprising a glucose anti-metabolite to the animal. In another embodiment, methods of enhancing longevity in an animal are described, the methods comprising administration of a composition comprising avocado extract, wherein the avocado extract comprises mannoheptulose. In yet another embodiment, methods of enhancing longevity in an animal are described, the methods comprising administration of a composition comprising mannoheptulose.

### CROSS-REFERENCE TO RELATED APPLICATIONS

This application is a continuation-in-part of application Ser. No. 09/950,052, filed Sep. 12, 2001, currently pending, which is a continuation-in-part of application Ser. No. 08/889,877, filed Jul. 8, 1997.

### FIELD OF THE INVENTION

The present invention relates to the use of glucose anti-metabolites to alter utilization of glucose or other energy sources and to mimic metabolic effects of caloric restriction for the purpose of enhancing longevity in an animal.

### BACKGROUND OF THE INVENTION

Biological theories have correctly predicted the finding that a restriction of caloric intake by food deprivation slows down certain undesirable cellular processes in laboratory animals, many associated with aging and age-related diseases.

In particular, caloric restriction has been shown to consistently extend the life span, delay onset and slow tumor progression, and retard physiologic aging in many systems. Indeed, research spanning more than sixty years has shown that caloric restriction is a nutritional intervention that consistently extends longevity in animals. See Weindruch and Walford, “The Retardation of Aging and Disease by Dietary Restriction,” Springfield, Ill.: Charles C. Thomas (1988); Yu, “Modulation of Aging Processes by Dietary Restriction,” Boca Raton: CRC Press (1994); and Fishbein, “Biological Effects of Dietary Restriction,” Springer, N.Y. (1991). These effects of caloric restriction on life span and tumorigenesis have been reported numerous times since the early studies of McKay. See McKay et al., “The Effect of Retarded Growth Upon the Length of Lifespan and Upon Ultimate Body Size,” J. Nutr., Vol. 10, pp. 63-79 (1935). Indeed, over the past two decades, a resurgence of interest in caloric restriction in gerontology has led to the general acceptance that this dietary manipulation slows physiologic aging in many systems. See Weindruch and Walford, “The Retardation of Aging and Disease by Dietary Restriction,” Springfield, Ill.: Charles C. Thomas (1988); Yu, “Modulation of Aging Processes by Dietary Restriction,” Boca Raton: CRC Press (1994); and Fishbein, “Biological Effects of Dietary Restriction,” Springer, N.Y. (1991).

Reductions in fasting glucose and insulin levels are readily measured biomarkers of caloric restriction. Calorically restricted rodents exhibit lower fasting glucose and insulin levels, and the peak glucose and insulin levels reached during a glucose challenge are reduced in those on caloric restriction. See Kalant et al., “Effect of Diet Restriction on Glucose Metabolism and Insulin Repsonsiveness and Aging Rats,” Mech. Aging Dev., Vol. 46, pp. 89-104 (1988). It is also known that hyperinsulinemia is a risk factor associated with several such disease processes, including heart disease and diabetes (Balkau and Eschwege, Diabetes Obes. Metab. 1 (Suppl. 1): S23-31, 1999). Reduced insulin levels and body temperature are two of the most reliable indicators of this altered metabolic profile (Masoro et al., J. Gerontol. Biol. Sci. 47:B202-B208, 1992); Koizumi et al., J. Nutr. 117: 361-367, 1987; Lane et al., Proc. Nat. Acad. Sci. 93:4154-4164, 1996).

Glucose anti-metabolites such as 2-deoxy-D-glucose are compounds related to glucose. However, due to structural differences from glucose such compounds block or inhibit certain aspects of carbohydrate metabolism and may therefore mimic the effects of caloric restriction (Rezek et al., J. Nutr. 106:143-157, 1972). These anti-metabolites exert a number of physiological effects, including reduction of body weight, decrease in plasma insulin levels, reduction of body temperature, retardation of tumor formation and growth, and elevation of circulating glucocorticoid hormone concentrations. (For a review see Roth et al., Ann. NY Acad. Sci. 928:305-315, 2001). These effects result from inhibition of carbohydrate metabolism.

As such, use of glucose anti-metabolites as components for the enhancement of longevity in animal species, for example through decreasing abnormalities of glucose metabolism, would be beneficial.

### SUMMARY OF THE INVENTION

The present invention relates to the use of glucose anti-metabolites to alter utilization of glucose or other energy sources and to mimic metabolic effects of caloric restriction. In particular, in one embodiment herein, the invention relates to a method of enhancing longevity in an animal comprising administration of a composition comprising a glucose anti-metabolite to the animal. In another embodiment, the invention relates to a method of enhancing longevity in an animal comprising administration of a composition comprising avocado extract, wherein the avocado extract comprises mannoheptulose. In yet another embodiment, the invention relates to a method of enhancing longevity in an animal comprising administration of a composition comprising mannoheptulose.

### DETAILED DESCRIPTION OF THE INVENTION

Various documents including, for example, publications and patents, are recited throughout this disclosure. All such documents are hereby incorporated by reference. The citation of any given document is not to be construed as an admission that it is prior art with respect to the present invention.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments or individual features are disclosed. As will be apparent to the ordinarily skilled practitioner, all combinations of such embodiments and features are possible and can result in preferred executions of the present invention.

The compositions herein may comprise, consist essentially of, or consist of any of the features or embodiments as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

The present invention relates to the use of glucose anti-metabolites to alter utilization of glucose or other energy sources and to mimic metabolic effects of caloric restriction. Without intending to be limited by theory, the present use of glucose anti-metabolites to alter glucose metabolism serves to lower the metabolic rate through inhibition of glucose as an energy source on the cellular level. Judicious use of compounds that block the normal metabolism of cellular glucose can result in changes in physiological function that are similar to those arising from caloric restriction. Caloric restriction has been consistently shown to extend longevity in animals. See Weindruch and Walford, “The Retardation of Aging and Disease by Dietary Restriction,” Springfield, Ill.: Charles C. Thomas (1988); Yu, “Modulation of Aging Processes by Dietary Restriction,” Boca Raton: CRC Press (1994); and Fishbein, “Biological Effects of Dietary Restriction,” Springer, N.Y. (1991).

In one embodiment herein, the invention relates to a method of enhancing longevity in an animal, the method comprising administration of a composition comprising a glucose anti-metabolite to the animal. In another embodiment, the invention relates to a method of enhancing longevity in an animal, the method comprising administration of a composition comprising avocado extract, wherein the avocado extract comprises mannoheptulose. In yet another embodiment, the invention relates to a method of enhancing longevity in an animal, the method comprising administration of a composition comprising mannoheptulose. As used herein, “enhancing longevity,” with reference to an animal, includes both qualitative and quantitative enhancements, including prolonging the life span of the animal, retarding the physiological aging process, and/or the like, and/or improving the quality of life by reducing incidence of disease, maintaining vitality, and/or the like.

The animal treated herein may include mammals, or even invertebrates such as for example insects (e.g., the fruit fly). Humans and companion animals are advantageously treated herein. As used herein, “companion animal” means a domestic animal. Preferably, “companion animal” means a domestic dog, cat, rabbit, ferret, horse, cow, or the like. More preferably, “companion animal” means a domestic dog or cat.

The glucose anti-metabolites which are useful herein include 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, and mannoheptulose. Mannoheptulose is preferred for use herein. Advantageously, mannoheptulose may be present in the recited compositions as a component of an avocado extract, or other enriched source of mannoheptulose.

The following non-limiting illustrations exemplify the various embodiments of the present invention:

Decreased Utilization of Glucose as Energy Source by 2-Deoxy-D-Glucose:

To mimic the effects of caloric restriction, glucose anti-metabolites are provided over an extended time period. Previous studies show that 2-deoxy-D-glucose should not be administered in high doses, since significant untoward side effects and toxicity have been observed. However, studies in rodents (Lane et al., J. Anti-Aging Med. 1 (4): 327-337 (1998)) have shown that long-term disruption of glucose metabolism using a lower dose of 2-deoxy-D-glucose can mimic some of the major metabolic hallmarks of caloric restriction, enhanced longevity, including reduced body temperature, weight loss, and lower fasting insulin levels.

In light of the above potential physiologic benefits of caloric restriction weighed against the negative aspects of metabolic inhibition by 2-deoxy-D-glucose, alternatives which act as anti-metabolites of glucose without the potentially harmful side effects are preferred for purposes of practicing the invention.

Decrease of Availability of Glucose to Cells by 5-Thio-D-Glucose:

5-Thioglucose, an analog of glucose, has (in vivo) more pronounced effects than 2-deoxy-D-glucose. The compound is believed to act mainly by inhibiting glucose uptake by the cells. The majority of 5-thioglucose (97%) injected into a rat has been found excreted unchanged in urine (Hoffman et al., Biochemistry 7, pp. 4479-4483 (1968)). 5-Thioglucose is remarkably non-toxic; LD₅₀ was measured to be 14 g/kg, by injection, in rats (Chen et al., Arch. Biochem. Biophys., 169, pp. 392-396 (1975)).

Since 5-Thioglucose seems to be excreted unchanged in urine, this compound presents certain advantages for chronic administration over 2-deoxy-D-glucose. Since 5-thioglucose inhibits glucose uptake, appropriate dosing can result in benefits associated with caloric restriction, including enhanced longevity.

Effects of 3-O-Methylglucose:

This analog of glucose, in contrast with 2-deoxy-D-glucose, is not metabolized (Jay et al., J. Neurochem. 55, pp. 989-1000 (1990)) and, thus, may provide certain advantages for use in chronic administration. In the context of this invention, 3-O-methylglucose can prevent utilization of glucose as an energy source as demonstrated by response to its administration in rats. The responses were about seven times weaker than those to 2-deoxyglucose.

Effects of Anhydrosugars: 1,5-Anhydro-D-Glucitol (Polygalitrol):

This compound is a non-reducing analog of glucose and is enzymatically converted to 1,5-anhydroglucitol-6-phosphate, albeit the conversion is less efficient than that of 2-deoxy-glucose (Sols et al., J. Biol. Chem., 210, pp. 581-595 (1954). 1,5-anhydroglucitol-6-phosphate is an allosteric (non-competitive) inhibitor of hexokinase, which catalyzes the first regulatory step of glycolysis (Crane et al., J. Biol. Chem., 210, pp. 597-696 (1954)). Furthermore, 1,5-anhydroglucitol-6-phosphate is a non-reducing analog and cannot be a substrate for the next step of glycolysis catalyzed by glucose-6-phosphate isomerase. Consequently, this analog could accumulate in cells and act as a very effective metabolic block to glucose utilization. Another advantage relating to its non-reducing character is that this compound cannot be incorporated into glycolipids, glycoproteins, and glycogen. Thus, its effects are specific to glycolysis and would not be expected to affect other metabolic processes or exert toxicity of some glucose anti-metabolites previously discussed.

Interestingly, this compound (or its phosphate) has been found in the human body. It was found to be present in cerebrospinal fluid of patients who had occasional high blood glucose (from diabetes and diseases of the kidney) in large enough concentrations to be detected in tests performed in normal clinical settings.

Use of 2,5-Anhydro-D-Mannitol and 2,5-Anhydro-Glucitol:

These compounds are non-reducing analogs of fructose. Fructose is an important component of food and fructose phosphates and diphosphate are intermediate products of glycolysis. Nevertheless, inhibition of metabolic events involving fructose and its phosphates by anhydrosugar analogs is difficult. Alpha and beta anomers of fructose, which spontaneously inter-convert, correspond to different anhydrosugars, to 2,5 anhydroglucitol and 2,5-anhydromannitol, respectively. Thus, only a few of the enzymatic conversions can be inhibited by a single compound. The 2,5-Anhydromannitol has been investigated in some detail. That compound is taken up by cells and converted into 2,5-anhydromannitol-1-phosphate. That phosphate is an analog of fructose-1-phosphate, but cannot be cleaved by the aldolase and, thus, the utilization of both glucose and fructose by cells is blocked. The 2,5-Anhydromannitol had been found to interfere in glucose formation and utilization in isolated rat hepatocytes (Riquelme et al., Proc. Natl. Acad. Sci. USA, 80, pp. 431-435 (1983)).

Decrease of Glucose Utilization as Energy Source by Ketoses:

Mannoheptulose is present in reasonable amounts in some foods (e.g., avocados contain up to 5% of mannoheptulose, by wet weight) and can be classified as a “generally recognized as safe” substance for human consumption. In studies of metabolism, doses of 10 grams of mannoheptulose were safely administered to humans orally. About 5% of the mannoheptulose ingested was reported to appear in urine after oral administration. The fate of the injected mannoheptulose has previously been investigated in rats: 66% was excreted unchanged, 29% was metabolized and, a day after the injection, 5% remained in the body (Simon et al., Arch. Biochem. Biophys, 69, pp. 592-601 (1957)).

### EXAMPLE 1

Preparation of Mannoheptulose-containing Supplement: Fresh avocados (Lula variety) were obtained from Fresh King Incorporated (Homestead, Fla.). The avocados were manually split open and the pits were removed and discarded. The remaining skin and pulp were ground through a Hobart Commercial Food Preparation machine (Serial No. 11-10410235) using a 12¼ sieve. The ground avocado was then transferred to an Edwards Freeze Drier (Super Modulyo Model, Crawely, Sussex, England). The freeze drier was set at −20° C. for the first 24 hours, −5° C. for the following 24 hours and 5° C. for the final 72 hours. Upon removal from the freeze drier, the meal was ground to a powder using a Straub Grinding Mill (model 4E, Philadelphia, Pa.). The avocado meal was analyzed and found to contain about 10.35% mannoheptulose, by weight of the meal. It should be noted that the amount of mannoheptulose found in avocados varies with the particular strain and state of ripeness, some avocados having little or no mannoheptulose.

### EXAMPLE 2

Administration of Mannoheptulose to Beagle Dogs: The use of mannoheptulose for purposes of enhancing longevity as a result of mimicking caloric restriction was tested in beagle dogs through measurement of insulin reduction. As has been discussed, and is now widely accepted in the art, insulin reduction is a hallmark of caloric restriction and therefore a suitable indicator.

A total of 12 beagles were utilized for the study were utilized for the study and were fed a standard commercial diet through the study period. Fasting blood samples were drawn 7, 6, 4, and 2 days prior to administration of mannoheptulose. The mannoheptulose was delivered to the dogs in the form of a freeze-dried avocado meal containing from about 10% to about 12% mannoheptulose, by weight of the meal. This preparation was adjusted to provide mannoheptulose doses of 2, 20, and 200 mg/kg body weight (MH-2, MH-20, MH-200, respectively). Fasting blood samples were collected 1, 3, 5, and 7 days after initiation of the administration of mannoheptulose.

Insulin levels were lowered by up to 35% in dogs which had received the avocado meal when compared to those dogs on similar diets which had not received meal with their diets. Those changes were similar to the decreases found in animals on caloric restricted diets. In contrast, plasma glucose concentrations of dogs fed the same standard diet which did not contain the avocado meal did not show such effects.

Without intending to be limited by theory, the mechanism by which insulin is reduced relates to the fact that glucose must be metabolized by the pancreas to stimulate insulin secretion (German et al., Proc. Nat. Acad. Sci., 90, 1781-1785 (1993)). Mannoheptulose is thought to inhibit glucokinase, the initial enzyme involved in glucose metabolism in pancreas and liver. Therefore, reduced insulin levels indicate that mannoheptulose has indeed inhibited glucose metabolism, thereby mimicking caloric restriction. This effect on glucokinase by mannoheptulose would indicate use of mannoheptulose directed at inhibition of tumor growth as an alternative to administration of 2-deoxy-D-glucose. See Board et al., Cancer Res., 55(15): 3278-3285 (1995). Mannoheptulose would present a safe alternative to 2-deoxy-D-glucose, since it would avoid some untoward effects seen when 2-deoxy-D-glucose is administered on a long-term basis.

The availability of glucose to cells can also be decreased using other dietary supplements than those specifically identified herein which have similar effect on metabolism of glucose that can result in an inhibition of glucose processing.

The methods of the invention may be practiced by administering a compound described herein orally or parenterally, though oral administration would be preferred. When lowering of tissue metabolism is desired, as an adjunct to treatment of trauma, the compounds may be administered intravenously.

Dosage will depend upon the agent used and will vary depending upon the size and condition of the animal to which the agent is to be administered. Dosage in the range of 0.0001 grams/kg to about 1 g/kg, per kilogram of the animal, is beneficial. Dosage at the lower range would be appropriate when using 2-deoxy-D-glucose in large animals. Higher dosage, particularly of compounds such as 5-thio-D-glucose or mannitol would be readily tolerated.

In addition to the effects of glucose anti-metabolites on insulin and related metabolism in dogs, mice fed a diet containing, for example, mannoheptulose, also exhibit reduced plasma insulin levels and slightly reduced body weight. Both of these endpoints are closely related to altered energy metabolism, similar to that elicited by dietary caloric restriction. Even more important from a fundamental metabolic standpoint, fruit flies fed mannoheptulose exhibit lifespan extension of nearly 50%, an effect comparable to that exerted by caloric restriction in a number of animal species. It is important to note that all of these studies employ control preparations of avocados, containing little or no mannoheptulose, so biological effects are not due to other nutritional components of this fruit.

What is claimed is:

1. A method of enhancing longevity in an animal, the method comprising administration of a composition comprising a glucose anti-metabolite to the animal.

2. The method according to claim 1 wherein the animal is a mammal.

3. The method according to claim 2 wherein the composition comprises amounts of the glucose anti-metabolite sufficient to raise the glucose level in the blood of the animal subsequent to administration of the glucose anti-metabolite.

4. The method according to claim 2 wherein the administration is oral.

5. The method according to claim 4 wherein the glucose anti-metabolite is 2-deoxy-D-glucose.

6. The method according to claim 4 wherein the glucose anti-metabolite is 5-thio-D-glucose.

7. The method according to claim 4 wherein the glucose anti-metabolite is 3-O-methylglucose.

8. The method according to claim 4 wherein the glucose anti-metabolite is an anhydrosugar.

9. The method according to claim 8 wherein the glucose anti-metabolite is 1,5-anhydro-D-glucitol.

10. The method according to claim 8 wherein the glucose anti-metabolite is 2,5-anhydro-D-mannitol.

11. The method according to claim 4 wherein the glucose anti-metabolite is mannoheptulose.

12. The method according to claim 4 wherein the composition comprises avocado extract, wherein the avocado extract comprises mannoheptulose.

13. The method according to claim 4 wherein from about 0.0001 grams to about 1 gram of the glucose anti-metabolite, per kilogram of the animal, is orally administered to the animal.

14. The method according to claim 13 wherein the mammal is a companion animal.

15. A method of enhancing longevity in an animal, the method comprising administration of a composition comprising avocado extract, wherein the avocado extract comprises mannoheptulose.

16. The method according to claim 15 wherein the administration is oral.

17. The method according to claim 16 wherein the animal is a mammal.

18. The method according to claim 17 wherein from about 0.0001 grams to about 1 gram of the mannoheptulose, per kilogram of the animal, is orally administered to the mammal.

19. A method of enhancing longevity in an animal, the method comprising administration of a composition comprising mannoheptulose.

20. The method according to claim 19 wherein the administration is oral.

21. The method according to claim 20 wherein the animal is a mammal.

22. The method according to claim 21 wherein the animal is a companion animal.

23. The method according to claim 22 wherein the mammal is a cat.

24. The method according to claim 22 wherein the mammal is a dog.

25. The method according to claim 21 wherein from about 0.0001 grams to about 1 gram of the mannoheptulose, per kilogram of the mammal, is orally administered to the mammal.
